SEQUENCE LISTING (1 sequence)

SEQ ID NO: 1 - DNA, 424 bp, Ceriporia lacerata
  Ceriporia lacerata ITS-5.8 rDNA
cgggttgtagctggcctttaacgaggtatgtgcacgcctggctcatccactctcaacctctgtgcactttatgtaagaaacggtgtaagccagctattcattagttggtaataagcctttcttatgtttactacaaacgcttcagttatagaatgtttactgtgtataacacaattatatacaactttcagcaacggatctcttggctctcgcatcgatgaagaacgcagcgaaatgcgataagtaatgtgaattgcagaattcagtgaatcatcgaatctttgaacgcaccttgcactccttggtattccgaggagtatgcctgtttgagtctcatggaattctcaacccctaaattttgtaatgaagtttattgggcttggacttgtaggttgtgtcggcttctattcaactcctctgaaaa

(19) United States
(12) Patent Application Publication
(10) Pub. No.: US 2014/0193454 A1
(43) Pub. Date: Jul. 10, 2014

(54) METHOD FROM PREPARING CERIPORIA LACERATA CULTURE EXTRACT AND PHARMACEUTICAL COMPOSITION FOR PREVENTION OR TREATMENT OF DIABETES AND DIABETIC COMPLICATIONS COMPRISING CERIPORIA LACERATA CULTURE EXTRACT AS ACTIVE INGREDIENT

(71) Applicant: Byoung Cheon KIM, Gimcheon-City (KR)

(72) Inventor: Byoung Cheon KIM, Gimcheon-City (KR)

(21) Appl. No.: 13/737,144

(22) Filed: Jan. 9, 2013

(51) Int. Cl. A61K 36/07

(52) U.S. Cl. CPC A61K 36/07
USPC 424/195.15; 435/256.8

(57) ABSTRACT

A method for preparing a Ceriporia lacerata mycelium culture extract and a pharmaceutical composition for preventing or treating diabetes and diabetic complications, contains a Ceriporia lacerata mycelium culture extract prepared by the above method. The Ceriporia lacerata mycelium culture extract prepared according to the preparation method of the invention has a higher content of exopolysaccharide than the extract prepared by the method described in the prior art document of the present inventors, and thus it can be used as the active material of pharmaceutical compositions and functional foods against diabetic diseases.

### BACKGROUND OF THE INVENTION

1. Field of the Invention

The present invention relates to a method for preparing a Ceriporia lacerata mycelium culture extract and a pharmaceutical composition for preventing or treating diabetes and diabetic complications, which contains a Ceriporia lacerata mycelium culture extract prepared by the above method.

2. Description of the Prior Art

Ceriporia lacerate is a kind of white-rotting fungus and performs co-metabolism (lignin decomposition) in order to use carbon sources such as cellulose and hemi-cellulose in the ecosystem.

The presence of Ceriporia lacerata was reported first in 2002, and for this reason, there have been only a very small number of studies on the industrialization of Ceriporia lacerata. With respect to the industrialization of Ceriporia lacerata, only two types of studies on the use of Ceriporia lacerata for soil contamination preventing and bleaching are known.

With respect to studies on the use of Ceriporia lacerate for foods and drugs, Korean Patent Registration No. 10-1031605 (entitled “method for preparing Ceriporia lacerata culture extract for prevention and treatment of diabetic disease and Ceriporia lacerata culture extract prepared thereby”) filed in the name of the present inventors is the only study in the world. However, the above patent document mentions only the effect of the extract against type 1 diabetes.

Diabetes therapeutic agents developed to date include blood glucose lowering agents and insulin injections, but these agents merely delay the progression of diabetes and are not significant as agents for preventing and treating diabetes.

A substance for arresting the progression of the diseases is required to be developed. In addition, a substance for either regenerating pancreatic beta-cells that regulate insulin secretion or promoting the regeneration is required to be developed.

Recent study results showed that the marker substance exopolysaccharide contained in a Ceriporia lacerata mycelium culture and a dried substance thereof is a very effective substance for blocking the progression of diabetes and promoting the regeneration of pancreatic beta-cells. Thus, there is an urgent need for studies on the identification of the structure of exopolysaccharide in Ceriporia lacerata and the discovery of an optimal culture method for increasing the content of the exopolysaccharide.

### SUMMARY OF THE INVENTION

Accordingly, the present invention has been made in order solve the above-described problems occurring in the prior art, and it is an object of the present invention to provide an improved method for preparing a Ceriporia lacerata mycelium culture extract, which can increase the content of exopolysaccharide.

Another object of the present invention is to provide a pharmaceutical composition for preventing or treating diabetes and diabetic complications, which contains, as an active ingredient, a Ceriporia lacerata mycelium culture extract prepared by the above method and having an increased content of exopolysaccharide.

To achieve the above objects, the present invention provides a method for preparing a Ceriporia lacerata mycelium culture extract, the method comprising the steps of: culturing Ceriporia lacerata mycelia in liquid, drying the culture to form powder; and preparing a solvent extract from the powder, wherein a medium for culturing the Ceriporia lacerata mycelia comprises 1-2 wt % of sugar, 0.2-1 wt % of glucose, 0.2-1 wt % of starch, 0.1-0.5 wt % of sorghum powder, 0.1-0.5 wt % of barley powder, 0.2-2 wt % of soy fluor, 0.05-0.1 wt % of magnesium sulfate (MgSO₄), 0.05-0.1 wt % of monopotassium phosphate (KH₂PO₄), 0.05-0.1 wt % of dipotassium phosphate (K₂HPO₄) and 92-98 wt % of water and has a pH of 4.5-6.0.

The culturing is preferably carried out under a blue LED light source.

The culturing is preferably carried out at a carbon dioxide concentration of 1,000-2,000 ppm.

The present invention also provides a pharmaceutical composition for preventing or treating diabetes and diabetic complications, which contains, as an active ingredient, a Ceriporia lacerata mycelium culture extract prepared by the above method and having an increased content of exopolysaccharide.

The diabetes may be type 2 diabetes.

The diabetic complications may be selected from the group consisting of hyperglycemia, atherosclerosis, microangiopathy, diabetic retinopathy and kidney disease.

### BRIEF DESCRIPTION OF THE DRAWINGS

FIGS. 1a and 1b shows the results of ITS-5.8S rDNA sequencing of a Ceriporia lacerata strain according to the present invention.

FIG. 2a is a graph showing the content of residual sugar as a function of pH and the kind of sugar in the culture of a Ceriporia lacerata strain, and FIG. 2b is a photograph showing culture products.

FIG. 3 is a graph showing the growth of mycelia and the content of exopolysaccharide as a function of the kind of sugar.

FIG. 4 is a graph showing the growth of mycelia and the content of exopolysaccharide as a function of the concentration of glucose.

FIG. 5 is a graph showing the growth of mycelia and the content of exopolysaccharide as a function of a nitrogen source.

FIG. 6 is a graph showing the growth of mycelia and the content of exopolysaccharide as a function of the concentration of soy fluor as a nitrogen source.

FIG. 7 is a graph showing the growth of mycelia and the content of exopolysaccharide as a function of trace elements.

FIG. 8 is a graph showing the growth of mycelia and the content of exopolysaccharide as a function of the concentration of the trace element MgSO₄.

FIG. 9 is a graph showing the growth of mycelia and the content of exopolysaccharide as a function of culture time in a 5-L fermenter.

FIG. 10 is a graph showing the results of measuring the molecular weight of exopolysaccharide in a purified culture.

FIG. 11 schematically shows an experimental process for analyzing the activity of a Ceriporia lacerata mycelium culture extract of the present invention against diabetes.

FIG. 12 shows the food intake of type 2 diabetes mice treated with a Ceriporia lacerata mycelium culture extract.

FIG. 13 shows the water intake of type 2 diabetes mice treated with a Ceriporia lacerata mycelium culture extract.

FIG. 14 shows the pattern of increase in bodyweight of type 2 diabetes mice by treatment with a Ceriporia lacerata mycelium culture extract.

FIG. 15 shows the states of the livers of a normal mouse, a type 2 diabetes mice and a mouse treated with a Ceriporia lacerata mycelium culture extract.

FIG. 16 is a graph showing blood glucose levels as a function of fasting time.

FIG. 17 is a graph showing blood glucose levels as a function of time after oral administration of glucose.

FIG. 18 is a graph showing blood glucose levels after sacrifice of mice fed with a Ceriporia lacerata mycelium culture extract.

FIGS. 19a and 19b are a graph and a micrograph, which show that a Ceriporia lacerata mycelium culture extract promotes adipocyte differentiation in a manner similar to insulin.

FIG. 20 is a graph showing the degree of adipocyte differentiation by a Ceriporia lacerata mycelium culture extract as a function of the presence or absence of insulin.

FIGS. 21a and 21b show insulin signaling in adipocytes by a Ceriporia lacerata mycelium culture extract.

FIG. 22 is a graph showing the expression level of GLUT4 in adipocytes by a Ceriporia lacerata mycelium culture extract.

### DETAILED DESCRIPTION OF THE INVENTION

Hereinafter, the present invention will be described in detail.

The present inventors have found that an marker substance having excellent effects of blocking the progression of diabetes and diabetic complications and promoting the regeneration of beta-cells is the exopolysaccharide of a Ceriporia lacerata mycelium culture extract, and the present inventors have developed a method for preparing a Ceriporia lacerata mycelium culture extract, which can increase the content of the exopolysaccharide, and a pharmaceutical composition for preventing or treating diabetes and diabetic complications, which contains the Ceriporia lacerata mycelium culture extract, thereby completing the present invention.

Therefore, the present invention provides a method for preparing a Ceriporia lacerata mycelium culture extract, the method comprising the steps of: culturing Ceriporia lacerata mycelia in liquid, drying the culture to form powder; and preparing a solvent extract from the powder, wherein a medium for culturing the Ceriporia lacerata mycelia comprises 1-2 wt % of sugar, 0.2-1 wt % of glucose, 0.2-1 wt % of starch, 0.1-0.55 wt % of sorghum powder, 0.1-0.5 wt % of barley powder, 0.2-2 wt % of soy fluor, 0.05-0.1 wt % of magnesium sulfate (MgSO₄), 0.05-0.1 wt % of monopotassium phosphate (KH₂PO₄), 0.05-0.1 wt % of dipotassium phosphate (K₂HPO₄) and 92-98 wt % of water and has a pH of 4.5-6.0.

The culturing is preferably carried out under a blue LED light source.

The culturing is preferably carried out at a carbon dioxide concentration of 1,000-2,000 ppm.

In a preferred embodiment of the present invention, the Ceriporia lacerata mycelium culture extract can be prepared by a method comprising the following steps:

(a) culturing Ceriporia lacerata mycelia to obtain a Ceriporia lacerata mycelium culture;

(b) vacuum-drying or freeze-drying the culture to form powder; and

(c) extracting the powder with one or more solvents selected from the group consisting of water, ethanol and methanol.

The liquid culture of Ceriporia lacerata mycelia in step (a) is performed by culturing Ceriporia lacerata mycelia in liquid to obtain exopolysaccharide. A medium composition for the liquid culture may comprise 1-2 wt % of sugar, 0.2-1 wt % of glucose, 0.2-1 wt % of starch, 0.1-0.5 wt % of sorghum powder, 0.1-0.5 wt % of barley powder, 0.2-2 wt % of soy fluor, 0.05-0.1 wt % of magnesium sulfate (MgSO₄), 0.05-0.1 wt % of monopotassium phosphate (KH₂PO₄), 0.05-0.1 wt % of dipotassium phosphate (K₂HPO₄) and 92-98 wt % of water.

Herein, the liquid culture is preferably performed at a temperature of 20 to 25 ID, a pH of 4.5-6.0, an illumination intensity of 0.5 LUX, an air injection rate of 0.5-1.5 kgf/cm² and a carbon dioxide concentration of 1,000-2,000 ppm for 8-13 days using a blue LED light source. Most preferably, the liquid culture is performed under the conditions of temperature of 22 ID, pH of 5, air injection rate of 1.0 kgf/cm² and carbon dioxide concentration of 1,500 ppm for 10 days, and in this case, the content of exopolysaccharide is the highest.

The parent strain that is used in step (a) is a strain obtained by culturing an excellent strain, stored in PDA medium at 4 ID, in a shaking incubator at 25° C. for 7-9 days using PDB medium in an Erlenmeyer flask. Herein, the amount of the mycelium to be introduced as an inoculum source is most preferably 0.5% of the solution to be incubated. Because an increase in the mycelium amount (%/100 ml) does not lead to an increase in the content of exopolysaccharide, the medium composition should have selective culture conditions, which maximize the content of exopolysaccharide and are not the best nutrient ratio and environmental conditions for the growth of mycelia.

The culture solution is separated into mycelia and an aqueous solution. The separation is performed by removing mycelia from the culture solution using a centrifuge and repeatedly purifying the remaining solution using a multi-sheet filter press and a vibrating membrane separator (PALLSEP), followed by irradiation with UV rays for 1 minute. This is because the presence of mycelia in the culture solution results in the change in the content of the active ingredient due to the growth of the mycelia.

In step (b), the mycelium culture prepared in step (a) is vacuum-dried or freeze-dried to form powder. When the drying is carried out at high temperature, a significant portion of the active ingredient can be lost. For this reason, the drying is carried out at a temperature of 40° C. or lower, preferably 30° C. or lower, for 48-96 hours. In addition, for the drying in step (b), a vacuum freeze dryer is preferably used compared to a vacuum dryer in which a relatively high evaporation temperature is set, and in this case, the change in the content of the active substance is minimized.

In step (c), the dried mycelia culture obtained in step (b) is extracted with a solvent, thereby preparing a Ceriporia lacerata mycelium culture extract containing exopolysaccharide according to the present invention.

In step (c), 5 g of the dried powder is sufficiently suspended in 100 mL of distilled water, and the suspension is centrifuged at 8,000 rpm for 20 minutes. A 2-3-fold amount of cold alcohol is added to the supernatant, and the solution was placed in a refrigerator at 4° C. and allowed to stand for 12 hours.

The supernatant in the solution which had been allowed to stand is centrifuged again at 8,000 rpm for 20 minutes, and the precipitate is recovered, thereby preparing crude exopolysaccharide. The extract is preferably vacuum-freeze-dried at 30° C. or lower.

The Ceriporia lacerata mycelium culture extract prepared according to the present invention as described above has significantly high contents of active ingredients effective for the treatment of steroid-induced diabetes, and thus has a very excellent effect of arresting and treating diabetes-related diseases and complications. More specifically, the Ceriporia lacerata mycelium culture extract according to the present invention contains exopolysaccharide, known to have anti-diabetic effects, in a very large amount of 3.00±0.03%/100 ml. In addition, the dried extract contains the exopolysaccharide in a very large amount of 5.00±0.02%/100 mg.

The present invention also provides a pharmaceutical composition for preventing or treating diabetes and diabetic complications, which contains, as an active ingredient, a Ceriporia lacerata mycelium culture extract prepared by the above method and having an increased content of exopolysaccharide.

The diabetes may be type 2 diabetes.

The diabetic complications may be selected from the group consisting of hyperglycemia, atherosclerosis, microangiopathy, diabetic retinopathy and kidney disease.

The pharmaceutical composition which contains, as an active ingredient, the Ceriporia lacerata mycelium culture extract prepared by the above method, may further contain a suitable carrier, excipient or diluent which is generally used.

For example, a powder formulation is prepared by mixing 200 mg of the extract, 100 mg of rice powder and 10 mg of talc and packing the mixture into an airtight bag.

For example, a tablet formulation is prepared by mixing 100 mg of the extract, 50 mg of rice powder, 10 mg of lactose and 2 mg of magnesium stearate and compressing the mixture into a tablet.

For example, a liquid formulation is prepared by mixing 100 ml of the extract, 5 g of isomerized sugar, a suitable amount of pine fragrance and a suitable amount of a preservative and packing the mixture in a brown bottle. In this case, the material resulting from step (a) may be used instead of the extract.

Hereinafter, the present invention will be described in further detail with reference to examples. It is to be understood, however, that these examples are for illustrative purposes and are not intended to limit the scope of the present invention.

### Examples

### 1. Preparation of Ceriporia lacerata Culture Extract

1-1. Preparation of Ceriporia lacerata Culture

Ceriporia lacerata was isolated from Quercus serrata and subcultured to obtain a parent strain which was then freeze-stored at −80° C. The stored strain was subcultured 2-3 times in PDA medium (87 plastic bulbs), and then a sufficient amount of a complete strain was selected and stored in a refrigerator at 4° C. until use. In addition, 600 ml of PDB medium was placed in an Erlenmeyer flask, and then a PDA culture strain was added thereto and shake-cultured for 8 days. Also, a liquid culture medium comprising 1.5 wt % of sugar, 0.5 wt % of glucose, 0.5 wt % of potato starch, 0.25 wt % of soy flour, 0.25 wt % of sorghum powder, 0.05 wt % of magnesium sulfate (MgSO₄), 0.05 wt % of monopotassium phosphate (KH₂PO₄), 0.05 wt % of dipotassium phosphate and 96.85 wt % of water was sterilized in a 800-L fermenter at 121° C. and at an air injection rate of 1.5 kgf/cm², and then cooled to 23 t, after which it was inoculated with 600 ml of the PBD culture strain to be used as a starter. Then, Ceriporia lacerata mycelia were liquid-cultured in the medium at a temperature of 23 t, an aeration rate of 0.5-1.5 kgf/cm² and a carbon dioxide concentration of 1,000-2,000 ppm for 10 days, thereby preparing a Ceriporia lacerata mycelium culture.

1-2. Preparation of Ceriporia lacerata Culture Extract

The prepared Ceriporia lacerata mycelium culture was freeze-dried using a vacuum freeze dryer at a temperature of 25° C. for 72 hours to form powder. 5 g of the dried powder was suspended sufficiently in 100 ml of distilled water and then centrifuged at 8,000 rpm for 20 minutes, and a 2-3-fold amount of cold alcohol was added to the supernatant. The resulting solution was placed in a refrigerator at 4° C. and allowed to stand for 12 hours. The supernatant in the solution which had been allowed to stand was centrifuged again at 8,000 rpm for 20 minutes, and the precipitate was recovered, thereby extracting a crude exopolysaccharide. The crude exopolysaccharide was dried in a freeze dryer for 72 hours, thereby obtaining a complete exopolysaccharide.

### 2. Optimization of Conditions for Liquid Culture of Ceriporia lacerata

2-1. Experimental Method

2-1-1. Culture Conditions

For optimization of Ceriporia lacerata liquid culture conditions according to shaking flask culture conditions, physiochemical characteristics and the mycelium and exopolysaccharide contents were measured using various kinds and concentrations of carbohydrates and micronutrients. For evaluation of characteristics, glucose, sucrose, lactose, fructose and galactose were used at a concentration of 3-5%, and nitrogen sources, tryptone, yeast extract, soy flour, L-glutamic acid, ammonium persulfate, malt extract and peptone were used as carbon sources at a concentration of 0.25%. As micronutrients, KH₂PO₄, MgSO₄, ZnSO₄, CuSO₄, FeSO₄ and CaCl₂ were used at a concentration of 0.1-0.5%. Culture was carried out in a 1,000 mL Erlenmeyer flask in a total volume of 800 mL at 25° C. and 120 rpm for 8 days, followed by analysis. In a 5 L jar fermenter, culture was carried out in a total volume of 3 L for various periods of time (days), thereby analyzing physicochemical characteristics as a function of culture time (days).

2-1-2. Measurement of pH, Acidity and Brix

pH was measured with a pH meter, and Brix was measured using an electronic Brix meter. Acidity was measured by measuring the pH of 10 mL of a culture with a pH meter, adding 0.1 N NaOH until the pH of the culture reached 8.3, and then calculating the consumption of 0.1 N NaOH to determine the content of tartaric acid.

2-1-3. Method for Measurement of Mycelium and Exopolysaccharide Contents

A culture was centrifuged at 12,000×g for 20 minutes, and the precipitate was washed three times with distilled water and then filtered. The filtrate was freeze-dried and weighed, thereby measuring the production of mycelia. A culture was centrifuged at 12,000×g for 20 minutes, and a 2-fold volume of cold isopropyl alcohol was added to the supernatant. Then, the solution was incubated at 4° C. overnight and centrifuged again at 12,000×g for 20 minutes, and the supernatant was dissolved in distilled water, after which it was freeze-dried and weighed, thereby measuring the production of mycelia.

2-1-4. Measurement of Tyrosine Content and Protease, α-Amylase and Fibrinolytic Enzyme Activities

In order to measure the production of peptide in a Ceriporia lacerata culture, the content of tyrosine in the culture was measured using folin phenol reagent. Specifically, 0.7 mL of 0.44 M TCA (trichloroacetic acid) was added to 0.7 mL of the culture which was then incubated at 37° C. for 30 minutes and centrifuged at 15,000 rpm for 10 minutes, and the precipitate was removed. 2.5 mL of 0.55 M Na CO₃ and 0.5 mL of phenol reagent were sequentially added to 1 mL of the collected supernatant and was allowed to react in a water bath at 37° C. for 30 minutes. The reaction solution was cooled to room temperature, and then the absorbance at 660 nm was measured using a spectrophotometer (UNION, Kontron Instruments, France).

The activities of α-amylase and protease in the culture were measured, and the enzyme activities were measured using the culture as an enzyme solution. As a substrate for α-amylase, 1 mL of 1% soluble starch (0.02 M phosphate buffer, pH 7.0) was used. 1 mL of a previously prepared enzyme solution was added to the substrate and allowed to react at 37° C. for 30 minutes, and the reaction was stopped with 10 mL of 1 M acetic acid. 2 mL of an iodide solution (0.005% I₂+0.05% KI) was added to the reaction solution, and the absorbance at 660 nm was measured. A decrease in blank OD of 10% was taken as 1 unit, and the result was expressed in terms of 1 g of the sample. As a control blank, a solution obtained by boiling a previously enzyme solution at 100° C. for 30 minutes was used.

To measure the activity of protease, 0.35 mL of 0.6% casein solution as a substrate and 0.35 mL of the enzyme solution were placed in an e-tube and allowed to react in a water bath 37° C. for 10 minutes, and then 0.7 mL of 0.44 M TCA solution was added thereto to stop the reaction. The reaction solution was allowed to stand at 37° C. for 30 minutes. The reaction solution was centrifuged at 15,000 rpm for 15 minutes, and then 2.5 mL of 0.55 M Na CO₃ and 0.5 mL of 3-fold-diluted folin reagent were added to 1 mL of the filtrate and then allowed to react at 37° C. for 30 minutes. Then, the absorbance at 660 nm was measured. The enzyme amount that liberates 1 g of tyrosine for 1 minute under such reaction conditions was taken as 1 unit.

The activity of fibrinolytic enzyme was measured using the Astrup and Mllerz method that is a kind of fibrin plate method. Specifically, on a fibrin plate, 10 mL of a solution of 0.5% fibrinogen in 0.067 M sodium phosphate buffer (pH 7.4) was added to a 9 cm-diameter Petri dish. To the solution, 0.1 mL of a solution of thrombin (100 units/mL) in 0.067 M sodium phosphate buffer (pH 7.4) was added and mixed rapidly, and the mixture was allowed to stand at room temperature for 30 minutes so as to be solidified. 20 L of the culture was dropped onto each marked position of the Fibrin plate and allowed to react at 37° C. for 2 hours, and then the enzyme activity was determined by the dissolution area. The standard curve of standard plasmin enzyme activity was plotted, and the fibrinolytic enzyme activity (%) of the culture was expressed as plasmin units in comparison with the standard curve. As a control, the purified fibrinolytic enzyme plasmin (5 units/mL) was used.

2-1-5. Measurement of Sugar and Protein Contents

The sugar content was measured by the phenol-sulfuric acid method. Specifically, the sugar content was determined by adding 25 μL of 80% phenol to 1 mL of a sample diluted at various concentrations, adding 2.5 mL of sulfuric acid thereto, cooling the mixture to room temperature, and measuring the absorbance at 425 nm. The protein content was measured by the BCA method using bovine serum albumin as a standard.

2-1-6. Measurement of Molecular Weight of Exopolysaccharide (EPS) by GPC

The dry viscous substance was dissolved in 0.1 M Na₂SO₄/0.05 M NaN₃ solution (adjusted to a pH of 4 with glacial acetic acid) at a concentration of 1%, and the solution was centrifuged. The supernatant was filtered through a 0.45 μm syringe filter and analyzed by GPC (Gel Permeation Chromatography) under the following conditions: detector: RI; GPC column: Shodex SB 805 HQ (Japan); mobile phase: 0.1 M Na₂SO₄/0.05 M NaN₃ (adjusted to a pH of 4 with glacial acetic acid); and flow rate: 1.0 mL/min. A standard curve was plotted using dextran (American Polymer Corporation, USA) having different molecular weights (130, 400, 770 and 1,200 kDa), and the molecular weight of EPS was measured using a refractive index meter (Table 1).

TABLE 1

| | Determination of Molecular weight |
|---|---|
| HPLC system | Knauer K-501 system |
| Column | OHpak SB 805 HQ (Shodex, Japan) |
| Mobile phase | 0.1M Na2SO4/0.05M NaN3/pH 4 |
| Flow rate | 1.0 mL/min |
| Detector | RI (Knauer K-2310) |

2-2. Experimental Results

2-2-1. Sequencing of Ceriporia lacerata ITS-5.8S rDNA

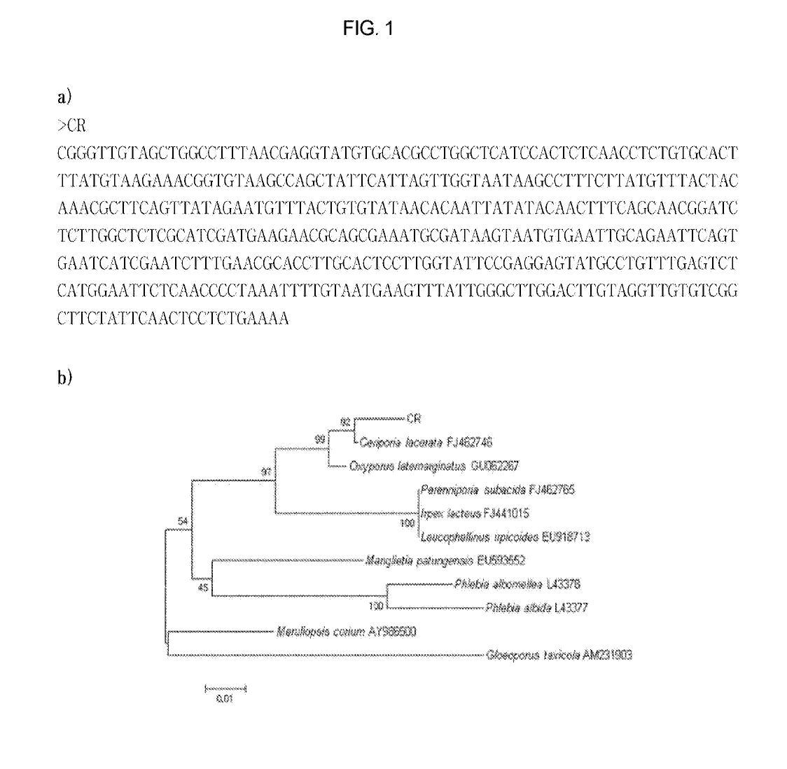

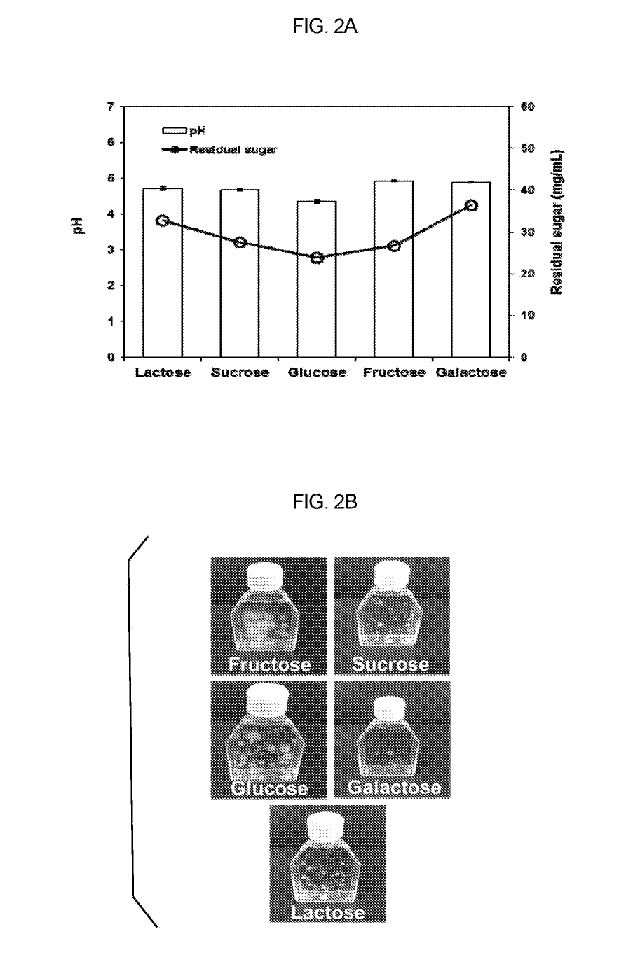

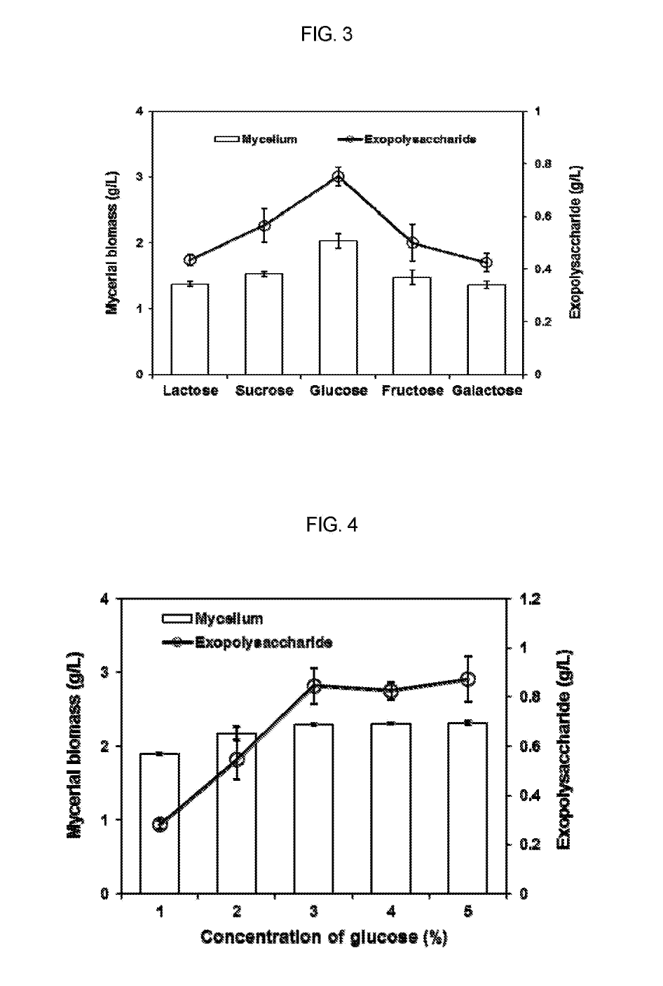

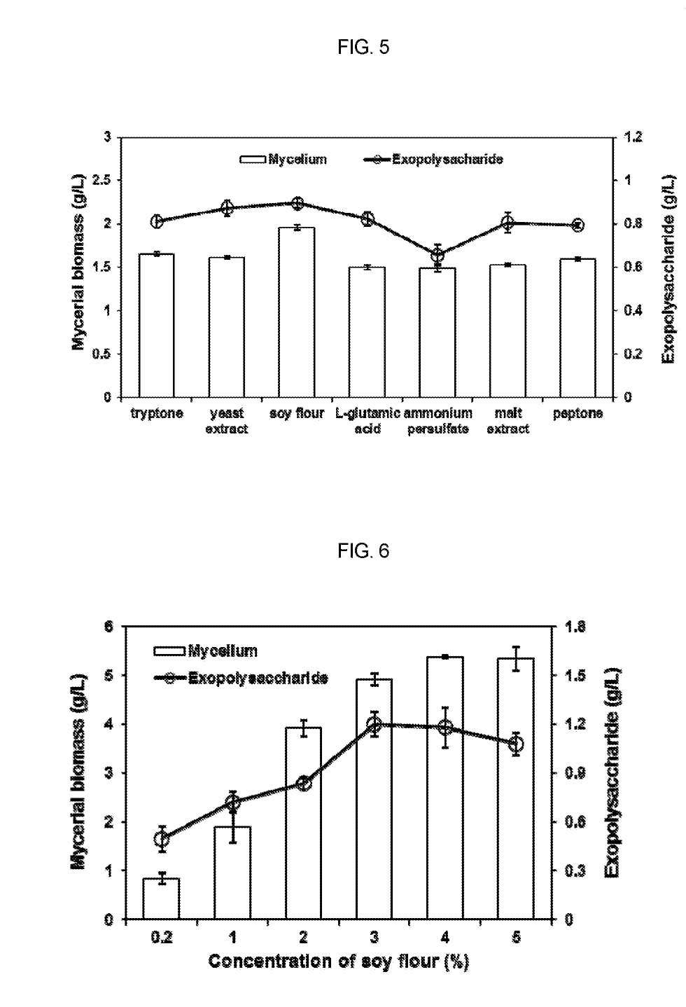

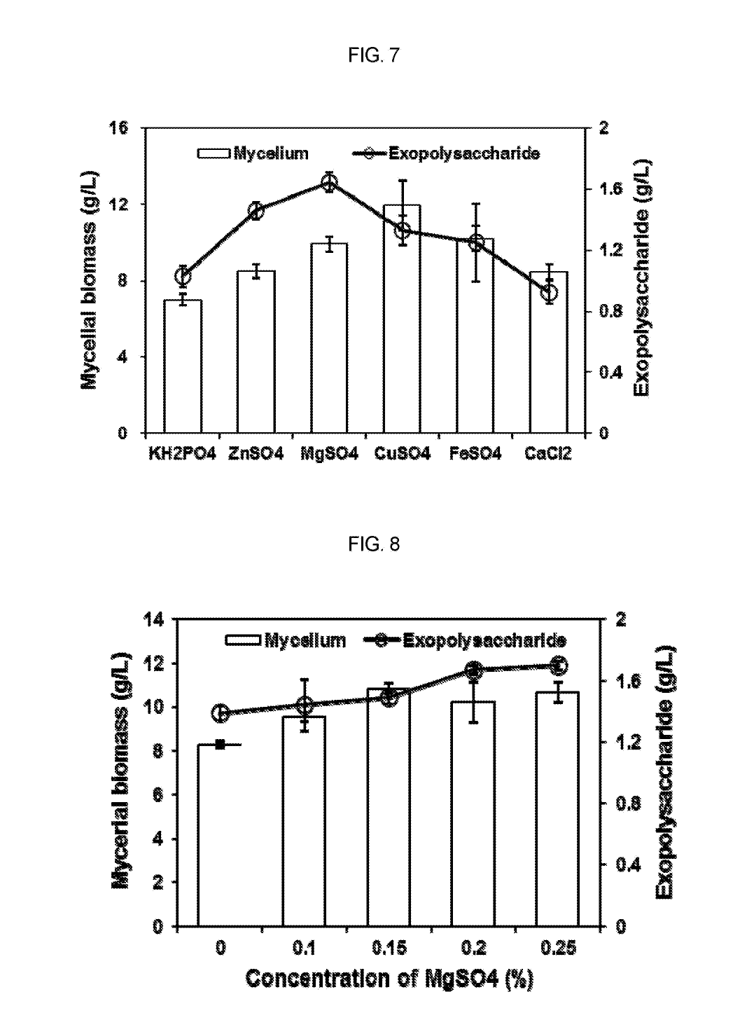

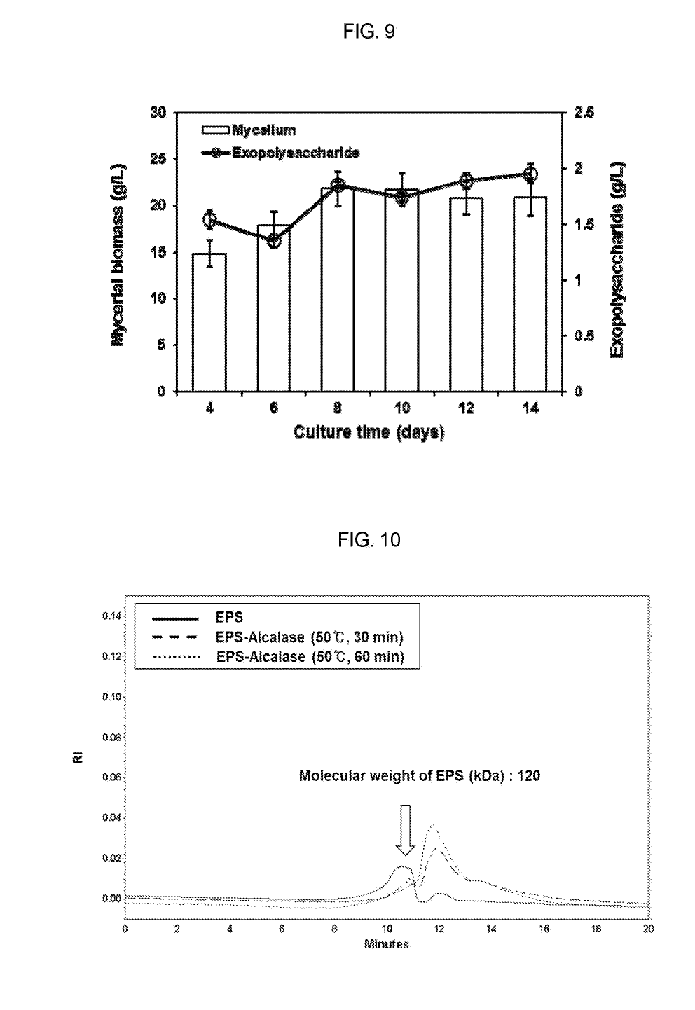

ITS-5.8S rDNA sequencing of the Ceriporia lacerata strain showed that the strain has a sequence homology of 92% with Ceriporia lacerata FJ462746 (FIG. 1).

2-2-2. Evaluation of Physiochemical Characteristics According to the Kind and Concentration of Sugar

To evaluate physiochemical characteristics according to the kind of sugar, each of five kinds of sugar (lactose, sucrose, glucose, fructose and galactose) was added at a concentration of 3% and incubated for 7 days. As a result, it was found that there was no great difference in pH between the sugars, and residual sugar was slightly low in the case of glucose. Also, it was shown that the shape and size of mycelial pellets changed depending on the kind of carbon source, suggesting that the influence of the carbon source on mycelial growth is very significant. Also, the mycelium and EPS contents were higher in the order of glucose, fructose and sucrose. Glucose was added at various concentrations up to 15%, and the mycelium and EPS content were measured. As a result, the contents increased in a concentration-dependent manner up to 3%, and then did not significantly change. Thus, a glucose concentration of 3% was chosen as an optimal condition.

2-2-3. Evaluation of Physiochemical Characteristics According to the Kind and Concentration of Nitrogen Source

In order to evaluate physiochemical characteristics according to the kind of nitrogen source, each of 7 kinds of nitrogen source (tryptone, yeast extract, soy flour, L-glutamic acid, ammonium persulfate, malt extract and peptone) was added at a concentration of 3% and incubated for 7 days. The mycelium content was the highest in the case of soy flour, and the EPS content was high in the cases of tryptone, yeast extract, soy flour and L-glutamic acid at similar levels. However, in economical and industrial terms, soy flour showing high mycelium and EPS contents was selected as a nitrogen source. When soy flour was added at a concentration of 0.25%, there was no great change in the pH of the culture, and the Brix of the culture increased in a manner dependent on the concentration of soy flour. The tyrosine content of the culture also increased in a manner dependent on the concentration of soy flour, and the protease and alpha-amylase activities were high at a soy flour concentration of 2˜3% and slightly decreased at higher soy flour concentrations. However, the fibrinolytic enzyme increased in a manner dependent on the concentration of soy flour. The mycelium and EPS contents showed a tendency to increase up to a soy flour concentration of 3% and did not significantly change at a soy flour concentration of more than 3%, like the case of the carbon source concentration. Thus, the optimal soy flour concentration was chosen to be 3%. The contents of sugar and protein in the EPS of the culture obtained by culture in the presence of soy flour were measured, and as a result, the sugar content was about 40% and the protein was about 33%, suggesting that the EPS is a polysaccharide composed of sugar bonded with protein. Table 2 below shows chemical characteristics and enzyme activities as a function of the content of soy flour, and Table 3 below shows the composition of exopolysaccharide as a function of the content of soy flour.

TABLE 2

| | | | | | | Fibrinolytic |
|---|---|---|---|---|---|---|
| | | | Tyrosine | Protease | α-amylase | enzyme |
| Soy | | | content | activity | activity | activity |
| flour | pH | °Brix | (mg %) | (unit/mL) | (unit/mL) | (unit/mL) |
| 0.2 | 4.25 | 3.0 | 7.46 | 0.04 | 5.77 | 0.60 |
| 1 | 4.47 | 3.9 | 30.12 | 0.17 | 3.64 | 0.60 |
| 2 | 4.59 | 4.8 | 48.37 | 0.75 | 6.78 | 0.75 |
| 3 | 4.74 | 5.5 | 64.21 | 1.02 | 1.68 | 0.85 |
| 4 | 4.91 | 6.3 | 69.39 | 0.94 | 1.10 | 0.75 |
| 5 | 4.84 | 7.0 | 82.32 | 0.75 | 0.60 | 1.25 |

TABLE 3

| | Total sugar content | Total protein |
|---|---|---|
| Soy flour | (%) | content (%) |
| 0.2 | 50.24 ± 1.06 | 33.13 ± 0.30 |
| 1 | 47.94 ± 0.15 | 32.49 ± 1.01 |
| 2 | 42.78 ± 0.08 | 37.91 ± 0.01 |
| 3 | 40.57 ± 0.68 | 33.34 ± 1.41 |
| 4 | 38.46 ± 0.09 | 34.34 ± 0.20 |
| 5 | 32.63 ± 0.30 | 36.20 ± 0.81 |

2-2-4. Evaluation of Physicochemical Characteristics According to the Kind and Concentration of Trace Element

In order to evaluate physicochemical characteristics according to the kind of trace element, each of five kinds of trace elements (MgSO₄, ZnSO₄, CuSO₄, FeSO₄, and CaCl₂) was added at a concentration of 0.5% and incubated for 7 days. As a result, the mycelium content was the highest in the case of CuSO₄, but the EPS production was the highest in the case of MgSO₄, and thus MgSO₄ was selected as a tract element. In addition, the trace amount was added at various concentration of 0-0.25%, and the mycelium and EPS contents were measured. As a result, the contents increased up to a trace element concentration of 0.15%, but did not significantly change at a concentration of 0.22%.

Thus, the optimal concentration of the trace element was chosen to be 0.15%.

2-2-5. Evaluation of Physicochemical Characteristics According to Culture Time (5 L Jar Fermenter)

Culture was performed in a 5-L jar fermenter using the selected optimal medium while the mycelium and EPS contents were measured at various points of time. As a result, the mycelium content did not significantly change up to 8 days of culture, but showed to decrease after 10 days of culture, and the EPS content showed a tendency to increase after 8 days, but this increase was not significant.

2-2-6. Evaluation of Exopolysaccharide (EPS) Characteristics

EPS was subjected to secondary purification and treated with protease, and the protein and sugar contents thereof were measured. As a result, purification of EPS showed a decrease in the sugar content and an increase in the protein. In addition, the molecular weight of EPS was measured by GPC, and as a result, the molecular weight of EPS was about 120 kDa and was slightly decreased by treatment with protease.

### 3. Verification of Anti-Diabetic Effects of CLD and EPS in Type 2 Diabetes Model

3-1. Experimental Method

3-1-1. Ceriporia lacerata Culture Extract

The Ceriporia lacerata culture extract used in this experiment was one prepared in the above section 1.

3-1-2. Method for Measurement of Mycelium and Exopolysaccharide Contents

The freeze-dried culture was prepared into a 10% solution which was then centrifuged at 8,000 rpm for 20 minutes, and the supernatant was isolated. A 4-fold volume of cold isopropyl alcohol was added to the isolated supernatant and incubated at 4° C. overnight. Then, the solution was centrifuged again at 10,000 rpm for 20 minutes, and the precipitate was collected and weighed to determine the content of the marker substance exopolysaccharide. It was shown that the major component of the Ceriporia lacerata culture was a carbohydrate, the crude carbohydrate content was about 79% and the crude protein content was about 15%.

3-1-3. Experimental Animal and Experimental Design

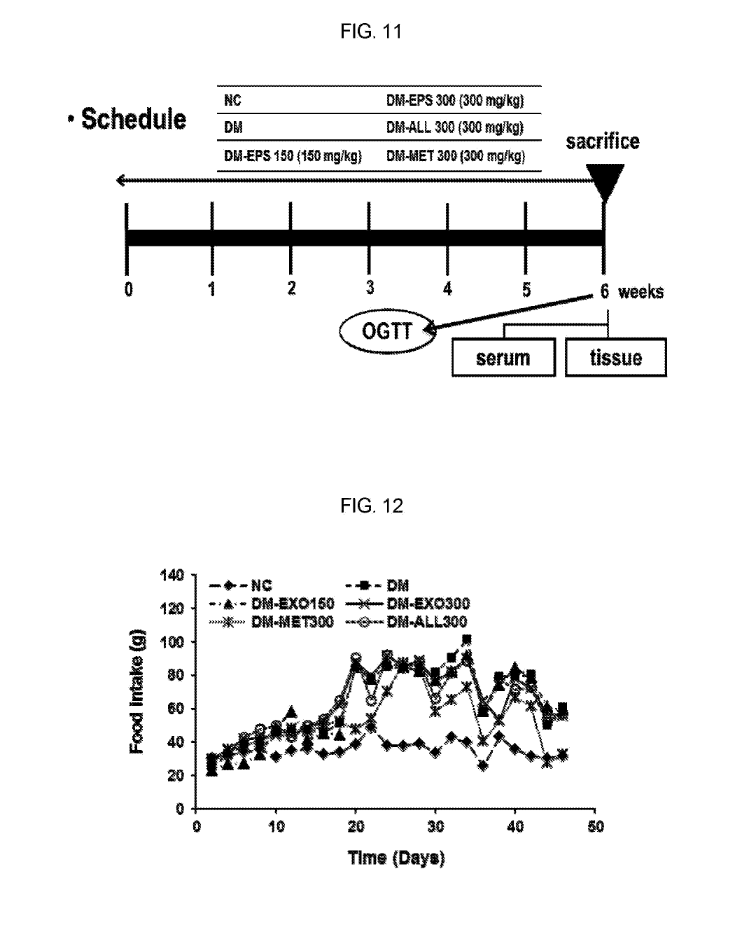

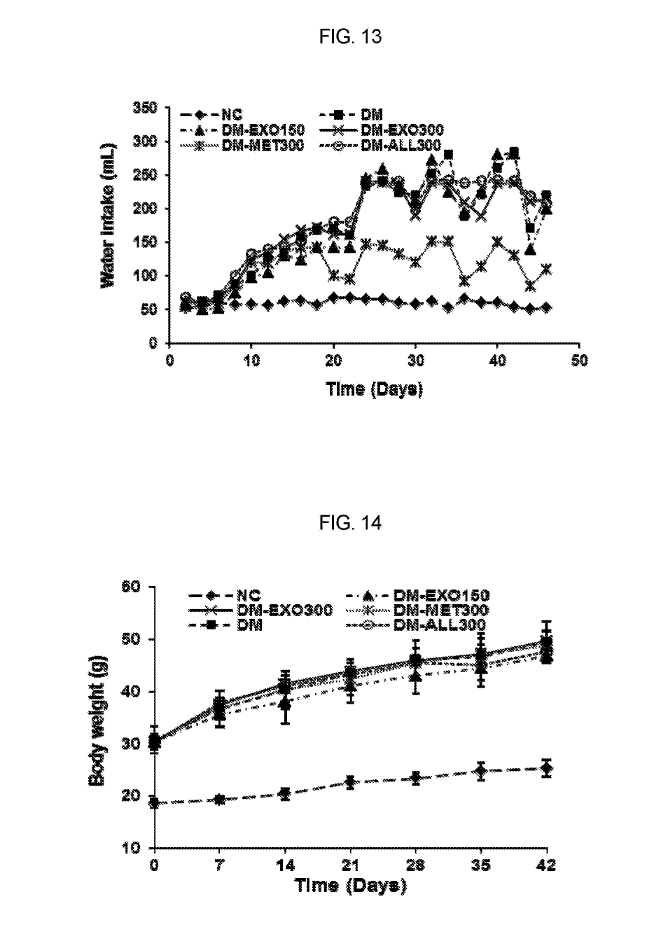

In order to examine the blood glucose lowering effects of the Ceriporia lacerata culture and exopolysaccharide, C57BL/Ksj (BL/Ls) homozygous diabetic (db/db) mice (SPF) as a typical type 2 diabetes animal model were used in this experiment. db/db mice are animals in which diabetes is caused by a point mutation in the leptin receptor gene Lepr of chromosome 4. In the animals, as the leptin receptor decreases, the signaling ability decreases, and thus the blood glucose level increases. The animals are recognized as an insulin-independent diabetes model and suitable for the evaluation and comparison of experimental results on the basis of raw data. For these reason, these db/db mice were selected in this experiment. The db/db mice used in this study were 6-week-old male mice weighing about 30-40 g, and these mice were produced in Japan SLC Inc. and obtained from Central Laboratory Animal (Korea). The mice were acclimated for about 7 days, and the weight and blood glucose level thereof were measured. 30 healthy animals suitable for experimentation and having no abnormal general conditions were selected. The experimental animals were divided according to a randomized block design into a negative control group, an exopolysaccharide low-dose group (150 mg/kg), an exopolysaccharide high-dose group (300 mg/kg) and a positive control group (metformin-300 mg/kg) such that the blood glucose level and weight are equal between the groups. These mice were reared for 6 weeks. In addition, normal and control groups, each consisting of 6 animals, were kept for 6 weeks under the same conditions. All test substances and positive control substances were orally administered at the same point of time every day, and the normal and negative control groups were orally administered with water (FIG. 11). During the rearing period of 6 week, a change in the weight was measured once weekly. In order to examine a change in the blood glucose level, the tail vein blood glucose level was measured using ACCU-CHEK Sensor (Germany) once weekly after 12-hr fasting. The experimental animals were fed with a commercially available animal solid feed (Samtaco Co. Ltd., Korea) and allowed access to water ad libitum. The animals were reared under the conditions of temperature of 23±3° C. and relative humidity of 50±10% with a 12-hr light/12-hr dark cycle (lighting: a.m. 8 to p.m. 8).

3-1-4. Oral Glucose Tolerance Test (OGTT)

The animals were fasted for 12 hours or more, and the fasting blood glucose level was measured. Each of samples obtained by dissolving each of the Ceriporia lacerata powder and EPS in distilled water at various concentrations was administered orally to five animals of each group. The control group was administered with the same amount of saline. Next, the groups other than the control group were orally administered with 40% glucose at a dose of 2 g/kg bw, and blood was collected from the tail vein at 30, 60, 90 and 120 minutes after glucose administration to observe a change in the blood glucose level. The increase in the blood glucose level at each point of time was calculated to plot a blood glucose curve.

3-1-5. Sacrifice of Experimental Animals and Sampling

Blood glucose levels should be measured in tail veins after 12-hr fasting, and blood for biochemical analysis should be collected after fasting of 12 hours or more. Thus, the sacrifice of the experimental animals was performed 2 days after 6^{th} blood glucose level measurement at 6 weeks after the start of rearing.

For sacrifice of the experimental animals, all the animals were fasted for 12 hours and anesthetized with ether. Blood was taken from the saphenous vein and placed in a tube for serum separation. Then, the blood was centrifuged at 3,000 rpm for 20 minutes to obtain serum which was to be used as a sample for analysis of biochemical markers. Immediately after sacrifice, the liver, abdominal fat and the like were extracted from all the animals, weighed, fast-frozen at −80° C. and stored until use. Portions of the extracted liver and pancreas were fixed in 4% paraformaldehyde solution and subjected to histological analysis.

3-1-6. Serum c-Peptide, Insulin and Leptin Levels

The serum c-peptide and insulin levels that are blood glucose-related functional indices were used in the serum (collected from the saphenous vein) using double antibody C-peptide (DPC, USA) using an insulin RIA kit (DPC, USA) and a mouse leptin RIA kit (LINCO, USA) by an radioimmunoassay.

3-2. Experimental Results

3-2-1. Changes in Food Intake and Weight

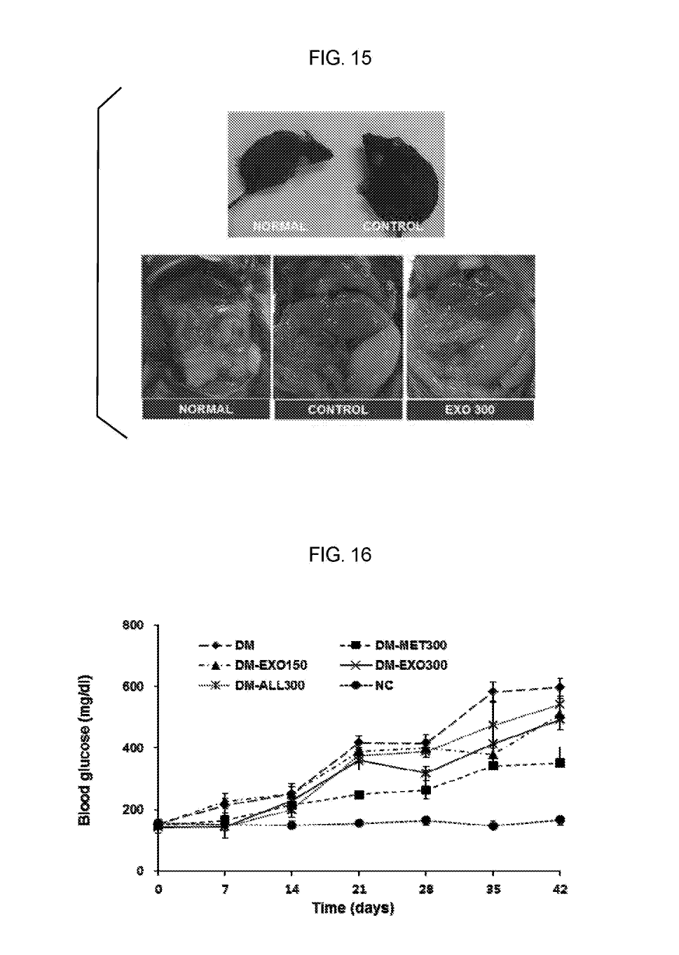

FIGS. 14, 15 and 16 show the changes in weight, food intake and water intake of the experimental animals for 6 weeks. The initial bodyweight of the diabetes control group and the initial bodyweight of the EPS-administered group were similar (about 32 g). Also, there was no significant difference in bodyweight after 6 weeks between the diabetes control group and the test groups, and the bodyweight showed a tendency to increase throughout the experimental period. The feed and water intakes were higher in the diabetes control group than in the normal control, and the water and feed intakes of the positive control group MET300 were significantly lower than those of other groups.

3-2-2. Organ Weight

The weights of liver, kidney, spleen, kidney fat and abdominal fat of the experimental animals were measured, and the results of the measurement are shown in Table 4 below.

TABLE 4

| | Liver | Kidney | Spleen | Kidney fat | Abdominal |
|---|---|---|---|---|---|
| | (W/BW) | (W/BW) | (W/BW) | (W/BW) | fat (W/BW) |
| NC | 0.94 ± 0.14 | 0.29 ± 0.03 | 0.06 ± 0.01 | 0.12 ± 0.04 | 0.43 ± 0.17 |
| DM | 2.89 ± 0.04 | 0.41 ± 0.06 | 0.04 ± 0.02 | 0.82 ± 0.17 | 2.34 ± 0.28 |
| DM-EXO150 | 2.50 ± 0.29 | 0.39 ± 0.05 | 0.06 ± 0.03 | 0.63 ± 0.12 | 2.48 ± 0.14 |
| DM-EXO300 | 2.65 ± 0.05 | 0.40 ± 0.04 | 0.05 ± 0.02 | 0.55 ± 0.17 | 2.55 ± 0.15 |
| DM-MET300 | 2.60 ± 0.19 | 0.39 ± 0.03 | 0.03 ± 0.02 | 0.89 ± 0.13 | 2.564 ± 0.33 |
| DM-ALL300 | 2.50 ± 0.27 | 0.40 ± 0.03 | 0.06 ± 0.01 | 0.48 ± 0.12 | 2.41 ± 0.21 |

The liver weight showed a tendency to increase rapidly in the diabetes-induced group and showed a tendency to significantly decrease in the groups administered with the sample. These results are consistent with the report that fat is accumulated in the liver upon the induction of diabetes to increase the liver volume. With respect to the kidney, it is known that the kidney volume increases with an increase in glomerular filtration rate in the initial stage of development of diabetes. In this study, the weight of the kidney also showed a tendency to increase, but this increase was not significant. In addition, the weight of the spleen did not significantly differ between the groups. The weight of the kidney fat increased rapidly in the diabetes-induced group, but was significantly decreased by administration of EPS and Ceriporia lacerata powder. In addition, the weight of the abdominal fat did not significantly differ between the groups.

3-2-3. Effect on Change in Blood Glucose Level

FIG. 16 shows the results of measuring the change in blood glucose levels after 12-hr fasting. The initial blood glucose level was similar (about 150 mg/dL) between the groups, but started to increase slightly from one week after administration of the sample. After 3 weeks, the blood glucose level increased rapidly so that the diabetes control group showed a blood glucose level of about 400 mg/dL, whereas the metformin group showed no increase in the blood glucose level. After that, the blood glucose level continued to increase, but the groups administered with EPS and Ceriporia lacerata powder showed a significant decrease in the blood glucose level compared to the diabetes control group.

3-2-4. Oral Glucose Tolerance

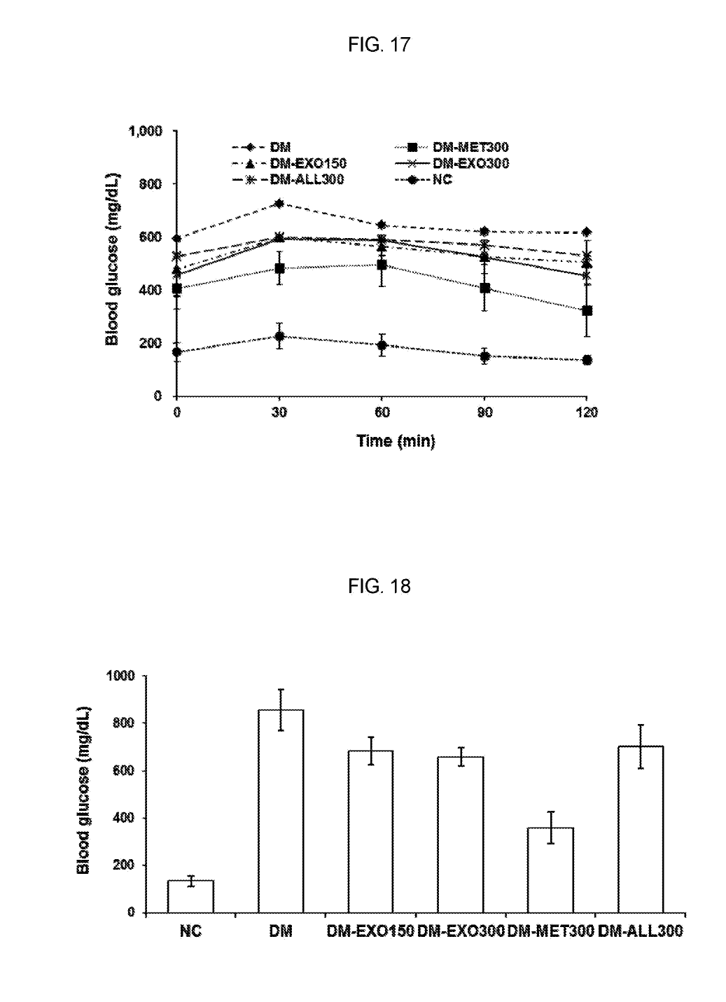

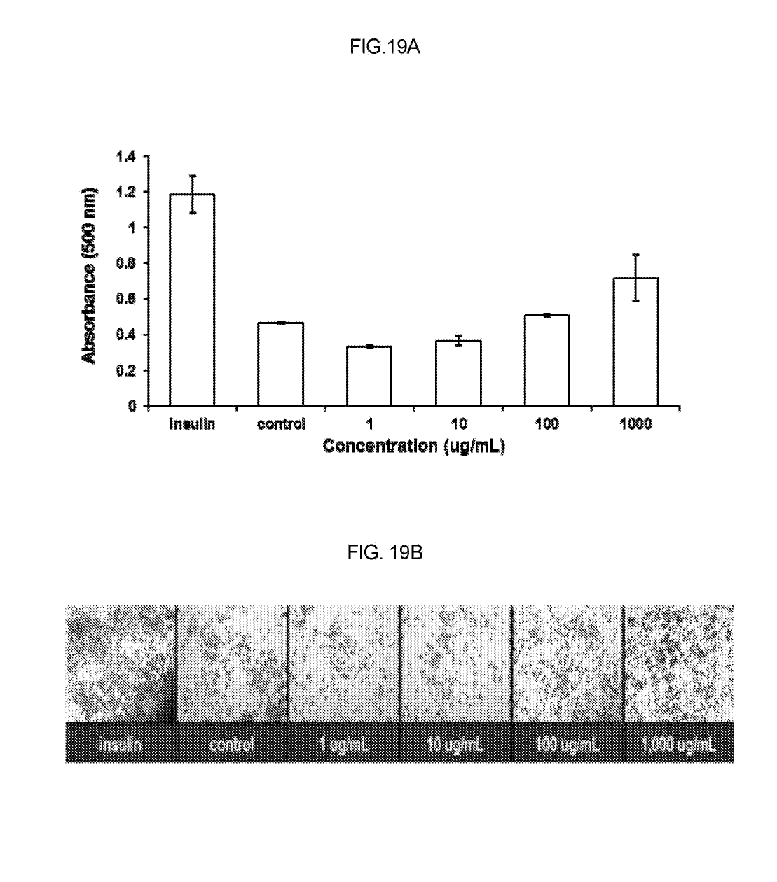

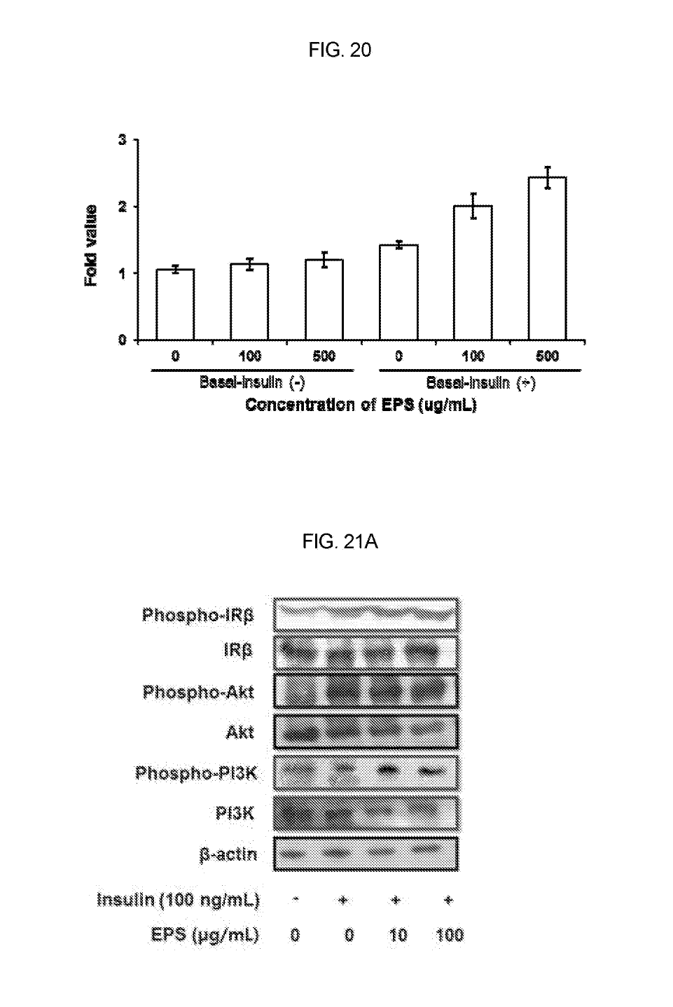

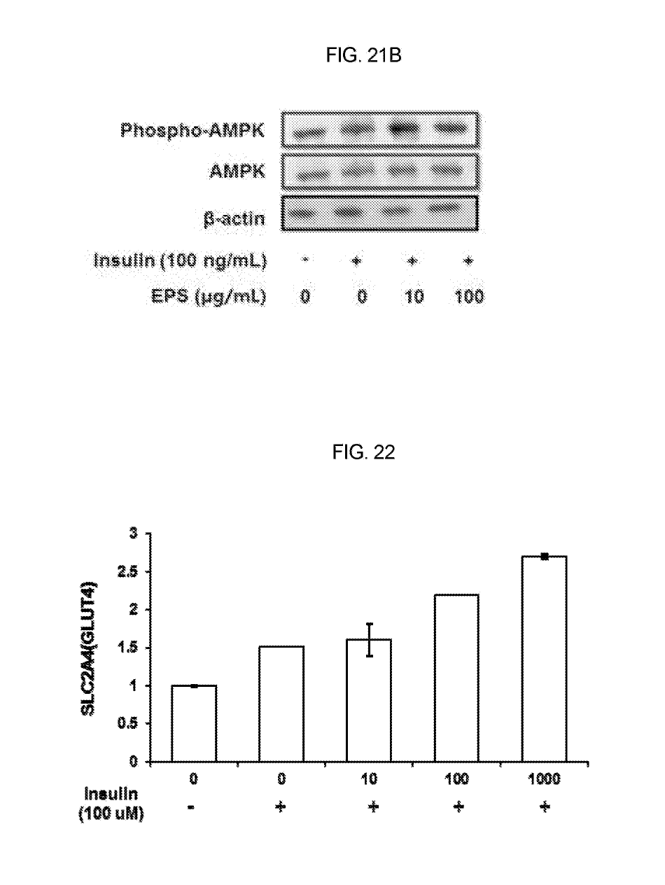

The oral glucose tolerance of EPS and Ceriporia lacerata powder was measured at 6 week of sample administration, and the results of the measurement are shown in FIG. 17. The diabetes control group showed a blood glucose level of 600 mg/dL which is the highest value measurable by the blood glucose meter, and it maintained high blood glucose levels throughout the glucose tolerance test. On the other hand, the group administered with EPS and Ceriporia lacerata powder showed an initial fasting blood glucose level of 500 mg/dL which was significantly lower than that of the diabetes control group. However, the blood glucose level of the group administered with EPS and Ceriporia lacerata powder increased gradually with the passage of time and was 600 mg/dL (the highest value measurable by the blood glucose meter) after 30 minutes. Thereafter, the blood glucose level gradually decreased, and after 180 minutes, was restored to 520 mg/dL which was similar to the initial blood glucose level.

3-2-5. Blood Glucose Level

The blood glucose levels of the animals sacrificed after 6-week oral administration of EPS were measured. As a result, it was shown that the blood glucose level of the DM group increased to about 900 mg/dL, whereas the blood glucose levels of the groups administered with the sample decreased in a manner dependent on the concentration of the sample. Specifically, the blood glucose level of the EPS 300 group decreased to about 700 mg/dL, suggesting that EPS plays a positive role in lowering blood glucose levels.

3-2-6. Effect on Serum Lipid Levels

The serum lipid levels of the animals sacrificed after 6-week oral administration of EPS were measured. As a result, it was shown that the total cholesterol and triglyceride levels were about two times higher in the DM group than in the NC group, but were significantly lowered in the groups administered with EPS. In addition, the groups administered with EPS showed a significant increase in the HDL cholesterol and a significant decrease in the LDL-cholesterol level, indicating that EPS is a substance that reduces serum lipid levels in type 2 diabetes models without changing the bodyweight.

3-2-7. Effects on Insulin, C-Peptide and Leptin Levels

The serum insulin, C-peptide and leptin levels of the animals sacrificed after 6-week oral administration of EPS were measured. As a result, it was shown that the insulin level did not significantly differ between the groups, and the C-peptide level was higher in the sample-administered groups than in the DM group, suggesting that EPS activates insulin secretion in the pancreas. In addition, the leptin level also increased in the sample-administered groups, suggesting that EPS plays a positive role in insulin resistance.

### 4. Effect of Exopolysaccharide on Insulin Signaling Mechanism in 3T3-L1 Cells

4-1. Experimental Method

4-1-1. Cell Culture

3T3-L1 fibroblasts that are preadipocytes have well established biological properties and differentiate into adipocytes when they are cultured under suitable conditions. These cells are used in studies on lipolysis inhibition or lipogenesis in the metabolic process of adipocytes. In addition, adipocytes that are insulin target cells are frequently used in studies on insulin signaling systems. The 3T3-L1 fibroblasts used in the experiment were obtained from the Korean Cell Link Bank and were cultured in Dulbecco's Modified Eagle's Medium (DMEM, GibcoBRL) (containing 10% fetal bovine serum (FBS, GibcoBRL), 200 mM glutaMAX (GibcoBRL), penicillin (10,000 units/ml, Sigma), streptomycin (10 mg/ml, Sigma)) under the conditions of 37° C. and 10% CO₂ while the medium was replaced with high-glucose medium at 3-day intervals. When the 3T3-L1 fibroblasts reached a confluence of 60-80%, the cultured cells were washed with Dulbecco's phosphate buffered saline (PBS, GibcoBRL), these cells were subcultured in a 75-cm² flask containing 500 μL of 2.5% trypsin (GibcoBRL) at 37° C. for 5 minutes, after which the cells were detached from the flask. The detached cells were transferred into a fresh flask containing 15 ml of a high-glucose DMEM medium supplemented with 10% FBS.

4-1-2. Glucose Uptake into Adipocytes

3T3-L1 fibroblasts were culture were cultured to confluence in the same manner as the above cell culture. 2 days after confluence, the cells were cultured for 3 days in a high-glucose DMEM medium containing 0.5 mM 3-isobutyl-1-methyl-xanthine (IBMX, Sigma), 25 μM dexamethasone (DEX, Sigma) and insulin (Sigma) which are differentiation inducers, and the medium was replaced with a fresh medium at 2-day intervals, whereby the cells were converted to adipocytes. Between 10 days and 15 days during which the preadipocytes were completely converted to adipocytes, a glucose uptake test was performed. In the glucose uptake test that reflects insulin sensitivity, the 3T3-L1 cells that had been completely converted to adipocytes were treated with 2.5% typsine, and then seeded into a 24-well plate at a concentration of 20×10⁴ cells/ml as counted by a hemacytometer. The medium was replaced with a low-glucose DMEM medium to starve the cells. The adipocytes in the well plate were washed with PBS and then incubated with a HEPS solution containing 0.1% bovine serum albumin (BSA, Roche), 10 μg/ml of the Ceriporia lacerata culture extract and 1 μg/ml of insulin at 37° C. for 1.5 hours. Then, 10 Ci/ml of the glucose analogue 2-deoxy-D-[3H] glucose (2-DG) was added to the cells which were then incubated at 37° C. for 10 minutes. After 10 minutes, the cells were washed five times with PBS, lysed with 1N NaOH and neutralized with 1N HCl, and the amount of 3H uptake into the cells was measured with a beta-counter (Tri-Carb 2100TR, Packard Bioscince, IL) for 5 minutes. In order to eliminate non-specific glucose uptake, cells incubated with cytochalasin B (Sigma) that inhibits the activity of glucose transporter 4 (GLUT4) were also measured. In the process of examining whether an insulin sensitive component is contained in the Ceriporia lacerata culture, low-concentration insulin was chosen to be 1 ng/ml and high-concentration insulin was chosen to be 25 ng/ml, because insulin uptake into the cells was the highest at an insulin concentration of 50 ng/ml. In this experiment, 1 ng/ml of insulin together with a Cordyceps militaris fraction was added to the 3T3-L1 adipocytes which were then incubated for 1.5 hours, after the glucose uptake into the cells was compared to the insulin uptake at 1 ng/ml. All the experiments were repeated three times. In order to eliminate the case in which an isolated substance acts as a detergent regardless of the action of insulin to destroy the cell membrane so as to increase glucose uptake, the glucose uptake of the isolated substance was measured together with a substance isolated at an insulin level of 0 ng/ml, and when the measured value was higher than the basal value, it was considered not to act as an insulin sensitive component, even it increased glucose uptake.

4-1-3. Examination of Expression of Protein Involved in Insulin Signaling System

For the experiment, 3T3-L1 adipocytes were treated with 2.5% typsine and transferred into a 24-well plate. 24 hours before the experiment, the cells were starved by replacement of the medium with a low-glucose Dulbecco's Modified Eagle's medium (DMEM) containing 10% fetal bovine serum. Then, the cells were washed with PBS and incubated with HEPES containing the Ceriporia lacerata culture extract and 1 ng/ml of insulin at 37° C. for 1 hour. Then, the cells were detached from the well plate on ice using RIPA buffer containing 50 unit aprotinin, 1 mM Na₃VO₄, and 1 mM PMSF. The detached cells were centrifuged at 10,000 rpm for minutes at 4° C. After centrifugation, the lower layer solution was diluted with Laemmli sample buffer, electrophoresed by SDS-PAGE and then transferred to a nitrocellulose membrane. The membrane was analyzed by Western blot using rabbit GLUT4 antibody (Chemicon, Temecula, Calif.), IR, PI3-Kinase, Akt, MAPK, AMPK antibody, and then the optical density was determined by a laser densitometer.

4-2. Experimental Results

4-2-1. Insulin Sensitivity

Insulin is a therapeutic agent which is mostly frequently used for treatment of type 1 diabetes and type 2 diabetes. Insulin is a factor that promotes adipocyte differentiation, and large amounts of adipocytes are produced by action of insulin. It was shown that the EPS of the Ceriporia lacerata culture extract promoted adipocyte differentiation in a concentration-dependent manner. It was found that the EPS is a natural material that can substitute for insulin.

4-2-2. Glucose Uptake

3T3-L1 fibroblasts that are preadipocytes have well established biological properties and differentiate into adipocytes when they are cultured under suitable conditions. Thus, these cells are used in studies on lipolysis inhibition or lipogenesis in the metabolic process of adipocytes. In this experiment, based on the fact that inducers such as insulin rapidly increase enzyme activity to promote differentiation, whether an insulin sensitive component is present was examined. Specifically, 3T3-L1 adipocytes converted by adding differentiation inducers such as insulin, IBMX and dexsamethasone were used in the experiment. Glucose uptake into the cells was determined by measuring the amount of the glucose analogue 2-deoxy-D-[3H]-glucose transferred into the cells by the glucose transporter GLUT4. For the glucose uptake test, 3T3-L1 adipocytes were starved with low-glucose DMEM before 24 hours and treated with HEPES and EPS. As a result, the glucose uptake into the cells by EPS could be seen. It was found that EPS acts as an insulin sensitive component to increase glucose uptake in a concentration-dependent manner, and this increase in glucose uptake was higher than the control basal value.

4-2-3. Expression of Protein Involved on Insulin Signaling System

The intracellular signaling of insulin occurs through various complex processes.

In the mechanism in which insulin acts on target cells, insulin shows various actions by binding to insulin receptor in the plasma membrane. Specifically, the insulin receptor is composed of an α-subunit and a β-subunit, and the action of insulin begins when insulin in blood binds to the α-subunit of the insulin receptor of target cells. The activated α-subunit activates tyrosine kinase of the β-subunit in the cell membrane. It is generally thought that the tyrosine kinase activity of the β-subunit is the initial stage of insulin action, which is necessary for many physiological actions of insulin.

When the tyrosine kinase of the β-subunit is activated, it phosphorylates IRS-1, IRS-2, IRS-3, IRS-4, Shc, p60 and the like which are proteins involved in the insulin signaling process, and then insulin signaling occurs through several down-signaling pathways with complex interactions. Among them, the tyrosine phosphorylation of IRS results in the activation of phosphatidylinositol 3-kinase (PI3-Kinase).

PI3-kinase is a heterodimer composed of a 110-kDa catalytic subunit and a 85-kDa regulatory subunit. IRS-1 and IRS-2 bind to p85 subunits of PI3-Kinase, and then activate p110 subunits to convert phosphatidylinositide-4,5-biphosphate to phosphatidylinositide-3,4,5-triphosphate. These phosphoinositides are thought to be signaling substances that play important roles in the biological action of various growth-stimulating factors, the exact function of each of the phosphoinositides in hormone signaling processes is not yet known. Through this PI3-Kinase pathway, p70S6kinase downstream thereof is activated, and kinase and various proteins undergo phosphorylation and dephosphorylation signaling processes. The activation of PI3-Kinase is important in many actions following insulin stimulation, including glucose movement, lipolysis inhibition, glycogen synthesis, protein synthesis and mitogenesis, but the relationship of PI3-Kinase with the occurrence of these responses is yet unclear.

Meanwhile, it is not thought that tyrosine kinase activation and tyrosine phosphorylation are always necessary for the action of insulin in all cells in all cases. It is known that there are signaling processes occurring regardless of tyrosine phosphorylation, and one of these signaling processes is a G protein signaling process. Among various G proteins, the GTP-binding protein Ras that induces various biological signals has been most actively studied. Ras is regulated by SOS and GTPase activating protein (GAP), and the activation of Ras sequentially induces the activation of MAP kinase (MAPKK), Raf-1 MAPK/E kinase (MAPKK or MEK), p90 ribosomal S6 kinase and the like. Also, among G proteins, ARF and Rho proteins appear to play an important role in the recirculation of a glucose carrier by activating phospholipase D, and Rab 4 protein is thought to play an important role in pathways related to the secretion of GLUT4. Some of such signaling pathways act independently or together to induce the expression of biological effects of insulin, including glucose transfer, enzyme activation, and synthesis of protein and nucleic acid. Insulin signaling processes include many signaling mechanisms and many protein substrates, and among them, IRS-1 plays a pivotal role in linking insulin signals, and IRS-1 appears to transfer the signal of insulin receptor to signaling substances such as PI3-kinase, GRB-2, SOS, Ras, Rab 4, ARF, SYP, Nck and the like. In this experiment, the effects of the Ceriporia lacerata mycelium culture extract that increases the glucose transporter GLUT4 on the insulin signaling system were examined. Specifically, the intracellular level of IRS-1 that plays a pivotal role in the insulin signaling system was examined in the presence of the Ceriporia lacerata mycelium culture extract, and the intracellular level of PI3-kinase that binds to IRS-1 to transfer the signal of insulin into cells was also examined. In addition, the intracellular level of GLUT4 that receives the signals of IRS-1 and PI3-Kinase to transfer glucose was also examined. As a result, it was found that all the proteins were phosphorylated by insulin and the expression levels thereof were increased by treatment with EPS. This suggests that EPS can promote glucose uptake through the IR, PI3K and Akt pathways and increase the expression of AMPK protein to promote glucose uptake, thereby improving insulin resistance.

### 5. Analysis of Marker Substance and Anti-Diabetic Functional Substance

5-1. Experimental Method

100 g of CLD was suspended in 1.5 L of water, and 1.5 L of hexane was added thereto. The suspension was placed in a reparatory funnel and fractionated into a hexane-insoluble layer and a hexane-soluble layer, and the upper layer was collected. Fractionation was repeated twice in the same manner as above using the same volume of hexane as the lower layer until the color of the solution became light so that the hexane-soluble substance was obtained in the largest possible amount. Subsequently, the fractions were combined and fractionated in the same manner as above using methylene chloride, ethylacetate and butanol, thereby obtaining 15 g of a hexane-soluble extract, 25 g of a methylene chloride-soluble extract, 30 g of an ethylacetate-soluble extract and 15 g of a butanol-soluble extract.

30 g of the ethyl acetate-soluble extract was subjected to silica gel column chromatography (12×60 cm, ASTM7734, Merck, Germany) using an eluent (hexane:ethyl acetate:methanol=10:3:1), and then fractionated by TLC, thereby obtaining 15 fractions.

5-2. 2,5-Dihydroxybenzoic Acid

Among the fractions, the 6^{th} fraction was separated by silica gel column chromatography using an eluent (toluene:ethyl acetate:acetic acid=5:3:1), and then purified by TLC (developing solvent/toluene:ethyl acetate:acetic acid=5:4:1), thereby obtaining 7 mg of a compound having an Rf of 0.58. The compound was analyzed by EI-MASS and ¹H-NMR, and as a result, the compound was identified to be 2,5-dihydroxybenzoic acid of the following formula:

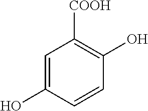 [SMILES: O=C(O)c1cc(O)ccc1O]

5-3. Separation of Protocatechualdehyde

Among the above fractions, the 7^{th} fraction was separated by silica gel column chromatography (1.5×15 cm, ASTM7734) using an eluent (toluene:ethylacetate:acetate=2.5:1:0.5), and then purified by TLC (developing solvent/toluene:ethylacetate:acetic acid=5:4:1), thereby obtaining 2 g of a compound having an Rf of 0.52. The compound was identified to be protocatechualdehyde of the following formula by EI-MASS and ¹H-NMR analysis:

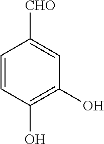

In order to measure the contents of the above two glucose tolerance compounds, LC/MS/MS analysis was performed using Agilent Technologies Agilent 6410 and a negative ion source at a fragmentor of 150 under the following conditions: gas temperature: 320° C.; gas flow rate: 35 mL/min; and capillary volt: 4000. The HPLC column used was Epic C18, and the temperature of the column was maintained at 40° C. The mobile phase used was composed of 0.1% formic acid-containing distilled water and 0.1% formic acid-containing acetonitrile.

As a result, 2,5-dihdroxybenzoic acid and protocatechualdehyde, known to have anti-diabetic effects, were detected in trace amounts, and the results of the analysis are shown in Table 5 below.

TABLE 5

| | | 2,5-dihdroxybenzoic |
|---|---|---|
| Sample | Protocatechualdehyde | acid |
| Example 5 | 18.79 ± 0.87 (μg/g) | 37.65 ± 1.32 (μg/g) |

5-3. Separation of Exopolysaccharide

In order to measure the content of exopolysaccharide reported to have excellent blood glucose lowering effects, 5 g of the Ceriporia lacerata mycelium culture extract was sufficiently suspended in 100 mL of distilled water, and the suspension was centrifuged at 8,000 rpm for 20 minutes. A 2-3-fold amount of cold alcohol was added to the supernatant which was then placed in a refrigerator at 4° C. and allowed to stand. The supernatant in the solution which had been allowed to stand was centrifuged again at 8,000 rpm for 20 minutes, and the precipitate was collected, thereby extracting crude exopolysaccharide. The crude exopolysaccharide was freeze-dried, and as a result, it was shown to have an exopolysaccharide content of 5.5±0.5%/100 mg. The sugar and protein contents of the EPS were measured, and as a result, it was shown that the sugar content was about 40% and the protein content was about 33%, suggesting that the EPS is a polysaccharide composed of sugar bonded with protein.

As described above, according to the present invention, there can be provided a Ceriporia lacerata mycelium culture extract containing 5.5±0.5%/100 mg of exopolysaccharide known to excellent blood glucose lowering effects, 18.79±0.87 μg/g of protocatechualdehyde and 37.65±1.32 μg/g of 2,5-dihdroxybenzoic acid.

### 6. Toxicity and Effect Tests in Rodents

Tests for the safety and effect of the Ceriporia lacerata mycelium culture extract were performed by K company (received a GLP certificate) according to Good Laboratory Practice (GLP). As a result, it was found that the Ceriporia lacerata mycelium culture extract statistically significantly increased pancreatic beta-cells in type 2 diabetes rats (increased the weight of pancreas, spleen and thymus) and was safer and more effective than Metform (Pfizer) used as a positive control drug (Table 6).

TABLE 6
general toxicity

| | |
|---|---|
| a single dose toxicity test on rodents | 7 weeks |
| toxisity test on rodents for 4 weeks repeat oral | 8 weeks |
| administration-DRF | |
| toxisity test on rodents for 13 weeks repeated oral | 27 weeks |
| administration(including recovered group) | |
| a single dose toxicity test on non rodents | 8 weeks |
| result | nontoxic judgement |

heredity toxicity test

| | |
|---|---|
| back mutation test(including preliminary test) | 4 weeks |
| chromosomal anomaly test(including preliminary test) | 8 weeks |
| micro nucleus test(including preliminary test) | 8 weeks |
| result | negative reaction |

effect test

| | |
|---|---|
| Oral Glucose Tolenance Test | 4 weeks |
| effect test on blood sugar dropping after being caused type 1 | 8 weeks |
| diabetes by STZ | |
| curing diabetes using db-db mice | 8 weeks |
| result | β-cell in pancreas with type 2 diabetes rats |
| | increased meaningfully in statistics, |
| | and it is judged that it is a fundamental diabetes |
| | medicine being recorded with safe and |
| | excellent effect compared to Metformin by |
| | pfizer used as a comparison medicine. |

As described above, the Ceriporia lacerata mycelium culture extract prepared according to the preparation method of the present invention has a higher content of exopolysaccharide than the extract prepared by the method described in the prior art document of the present inventors, and thus it can be used as the active material of pharmaceutical compositions and functional foods against diabetic diseases.

What is claimed is:

1. A method for preparing a Ceriporia lacerata mycelium culture extract, the method comprising the steps of: culturing Ceriporia lacerata mycelia in liquid, drying the culture to form powder; and preparing a solvent extract from the powder,
wherein a medium for culturing the Ceriporia lacerata mycelia comprises 1-2 wt % of sugar, 0.2-1 wt % of glucose, 0.2-1 wt % of starch, 0.1-0.5 wt % of sorghum powder, 0.1-0.5 wt % of barley powder, 0.2-2 wt % of soy fluor, 0.05-0.1 wt % of magnesium sulfate (MgSO₄), 0.05-0.1 wt % of monopotassium phosphate (KH₂PO₄) 0.05-0.1 wt % of dipotassium phosphate (K₂HPO₄) and 92-98 wt % of water and has a pH of 4.5-6.0.

2. The method of claim 1, wherein the culturing is carried out under a blue LED light source.

3. The method of claim 1, wherein the culturing is carried out at a carbon dioxide concentration of 1,000-2,000 ppm.

4. A pharmaceutical composition for preventing or treating diabetes and diabetic complications, which contains, as an active ingredient, a Ceriporia lacerata mycelium culture extract prepared by the method of claim 1.

5. The pharmaceutical composition of claim 4, wherein the diabetes is type 2 diabetes.

6. The pharmaceutical composition of claim 4, wherein the diabetic complications are selected from the group consisting of hyperglycemia, atherosclerosis, microangiopathy, diabetic retinopathy and kidney disease.
